(19) United States
(12) Patent Application Publication
(10) Pub. No.: US 2021/0085903 A1
(43) Pub. Date: Mar. 25, 2021

(54) COMBINATION MOUTHPIECE AND CHEEK SUPPORT FOR USE IN PERFORMING FORCED OSCILLATION TECHNIQUE

(71) Applicant: 12th Man Technologies, Inc., Garden Grove, CA (US)

(72) Inventors: Alex Stenzler, Long Beach, CA (US); Joanne Kay Creighton, Huntington Beach, CA (US)

(21) Appl. No.: 17/030,479

(22) Filed: Sep. 24, 2020

Related U.S. Application Data
(60) Provisional application No. 62/905,801, filed on Sep. 25, 2019.

(51) Int. Cl. A61M 16/04

(52) U.S. Cl. CPC A61M 16/0493; A61M 2205/02; A61M 16/047

(57) ABSTRACT

A mouthpiece for interfacing a subject to a respiratory measurement apparatus incorporates bite blocks and a tongue depressor such that when the bite blocks are clamped between teeth of the user's upper and lower jaws, the tongue depressor forces the user's tongue downward so as not to inhibit gas flow through a lumen of the mouthpiece. Also disclosed are cheek supports that can be coupled to the mouthpiece and where the space between adjacent cheek supports is selectively adjustable to fit the subject involved.

### CROSS-REFERENCED TO RELATED APPLICATIONS

This application is a non-provisional application of Application No. 62/905,801, filed Sep. 25, 2019, and claims priority from that application which is also deemed incorporated by reference in its entirety in this application.

### STATEMENT REGARDING FEDERALLY SPONSORED RESEARCH OR DEVELOPMENT

Not applicable

### BACKGROUND OF THE INVENTION

### I. Field of the Invention

The present invention relates generally to systems for diagnosing chronic obstructive pulmonary disease (COPD) and other lung diseases using the forced oscillation technique (FOT) and other lung function tests and, more particularly, to a patient mouthpiece especially designed to enhance the patient interface of the system and cheek supports that are designed for optional use with the mouthpiece.

### II. Discussion of the Prior Art

COPD is currently the fourth-most frequent cause of death in humans. The spirometry test is typically used to evaluate respiratory obstruction in COPD. Although considered the gold standard test, spirometry has the disadvantage of requiring great effort and great cooperation from patients, which can result in variation in the quality of test results and performing the test on some patients can be difficult, especially with infants.

In U.S. Pat. No. 5,318,038 to Jackson et al, there is described an infant respiratory impedance measuring apparatus in which the power amplifier is used to drive a loud speaker to superimpose a pulsatile variation in the pressure of air being breathed by a patient. The expiratory flow and pressure signals from transducers are amplified, filtered and applied to a program computer for analysis.

In the Jackson '038 patent to interface the hardware components to the patient, a mask covering the nose and mouth of the patient is employed.

It has been found that masks of the type described may lead to inaccuracies in the computation of lung impedance due to its excessive dead space. As is recognized in the prior art, a mouthpiece should be shaped so as to allow a patient to maintain a relatively airtight seal between the lips and the exterior thereof and that it could include a disposable filter to inhibit cross-contamination between patients of bacterial or viral infections. It is also important that the patient not obstruct the airflow with their tongue or clenched teeth.

It is accordingly a principle object of the present invention to provide an improved mouthpiece for use with equipment for measuring the respiratory impedance of a patient's lungs.

It has also been found that distortion of a subject's cheeks due to the pressure impulses imparted by the audio speaker and superimposed on the patient's airflow can adversely impact the accuracy of the impedance measurement in that the oral cavity will function as a shunt of the pressure and alter the results.

In the system illustrated, the speaker produces pressure waves which become superimposed with the air flows as the patient inhales and exhales. Pressure and flow transducers measure changes in response to the pressure waves introduced into the respiratory airflow, as well as changes in the flow rate of airflow. From this, the mechanical impedance (Z) of the patient's lung can be computed as the ratio of pressure (P) to flow (V) or Z=P/V. The speaker can be driven at various frequencies during the test and the computation of the impedance is made for each.

### SUMMARY OF THE INVENTION

The foregoing advantages over the prior art are achieved by providing an improved patient mouthpiece consisting of an integrally molded, silicone or elastomer rubber device having a round tubular portion that is designed to fit over a tubular or oval input/output member of a respiratory antibacterial filter device. Projecting out from the exterior wall of the tubular portion is a pair of wedge-shaped protuberances to which is attached proximally to a pair of laterally spaced, generally rectangular bite blocks. Completing the device and joined to the bite blocks by downwardly, and inwardly slanted bridge members is a generally rectangular tongue depressor.

The design is such that when the bite blocks are placed between a persons teeth on his or her upper and lower jaw, it prevents the teeth from closing in front of the lumen of the tubular portion and, the tongue depressor resides atop the person's tongue, preventing it also from partially occluding the lumen of the tubular portion of the mouthpiece and thereby altering the airway resistance. At the same time, the wedge members serve to create a seal with the natural shape of the subject's lips.

An attachment is provided for the mouthpiece that serves to stabilize the subject's cheeks. A cheek stabilizer is found to be important when conducting the FOT test procedure. The attachment comprises a toroidal ring designed to fit about and be supported on the tubular portion of the mouthpiece and provides support to a pair of cheek-engaging members that are releasably coupled to the toroidal ring at diametrically located positions. Cheek-engaging members can be joined to the ring at a plurality of locations so as to afford a selection of spacings between the cheek-engaging members, thereby accommodating a variety of patient's face dimensions.

### BRIEF DESCRIPTION OF THE DRAWINGS

The foregoing features, objects and advantages of the invention will become apparent to those skilled in the art from the following detailed description of the preferred embodiment, especially when considered in conjunction with the accompanying drawings in which like numerals in the several views refer to corresponding parts:

FIG. 1 is an enlarged perspective view of the mouthpiece of the present invention joined to an antibacterial respiratory filter;

FIG. 2 is a drawing of the mouthpiece alone;

FIGS. 3A through 3C are views illustrating the construction of cheek supports adapted for use with the mouthpiece of FIG. 1 when executing the forced oscillation technique test; and

FIG. 4 is an alternative embodiment of the cheek support.

### DESCRIPTION OF THE PREFERRED EMBODIMENT

This description of the preferred embodiments is intended to be read in connection with the accompanying drawings, which are to be considered part of the entire written description of this invention. In the description, relative terms such as “lower”, “upper”, “horizontal”, “vertical”, “above”, “below”, “up”, “down”, “top” and “bottom” as well as derivatives thereof (e.g., “horizontally”, “downwardly”, “upwardly”, etc.) should be construed to refer to the orientation as then described or as shown in the drawings under discussion. These relative terms are for convenience of description and do not require that the apparatus be constructed or operated in a particular orientation. Terms such as “connected”, “connecting”, “attached”, “attaching”, “join” and “joining” are used interchangeably and refer to one structure or surface being secured to another structure or surface or integrally fabricated in one piece, unless expressively described otherwise.

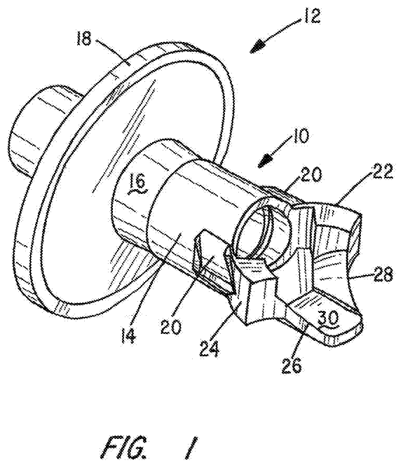

In FIG. 1 there is shown the mouthpiece member of the present invention indicated generally by numeral 10. It is shown connected to a respiratory hygienic filter indicated by numeral 12. Without limitation, the respiratory filter 12 could be of the type commercially available from MGC Diagnostics, Inc. of Vadnais heights, Minn., and designated as its Prevent® II Filter.

Mouthpiece 10 is seen to comprise a molded plastic member, preferably, but not necessarily, of a silicone rubber and having a tubular portion 14 with a circular cross section whose ID is sized to receive a tubular member 16 of the filter 12 therein. Tubular member 12 is integrally molded with a case 18 containing an anti-bacterial filter media specifically designed to permit gas flow therethrough while capturing any particulate matter that may be present in the air stream being inhaled and exhaled by a person undergoing FOT test.

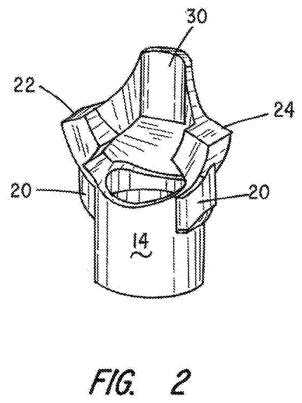

Integrally molded with and extending laterally outward from an outer wall of the tubular portion 14 is a pair of wedge-shaped projections 20. The shape of the wedge-shaped projections 20 can best be seen in the detailed drawing of FIG. 2. They are generally circumferentially spaced from one another by an arc of 180°.

The wedge-shaped projections 20 are integrally joined with a corresponding pair of cylindrical block segments as at 22 and 24. Block segments 22 and 24 are of a predetermined height and thickness dimension as seen in FIG. 2. Such dimensions are greater than is the thickness of the tubular portion 14.

Suspended from the blocks 22 and 24 by flexible bridge segments 26 and 28 (FIG. 1) that slope downwardly and inwardly is a generally rectangular portion 30.

With continued reference to FIG. 1, when the proximal portion of the mouthpiece is inserted into a subject's mouth with the tubular portion 14 extending out therefrom, and with the block members 22 and 24 clamped between the subject's upper and lower teeth, it prevents the teeth from closing in front of the tubular portion and, the generally rectangular portion 30, when resting upon the upper surface of the tongue, will tend to depress same against the floor of the mouth so as not to partially block airflow through the lumen of the tubular section 14.

The wedge-shaped projections 20 on the mouthpiece function to help seal the subject's lips at the corners of the subject's mouth when the subject has clamped the bite blocks 22 and 24 between his or her jaws.

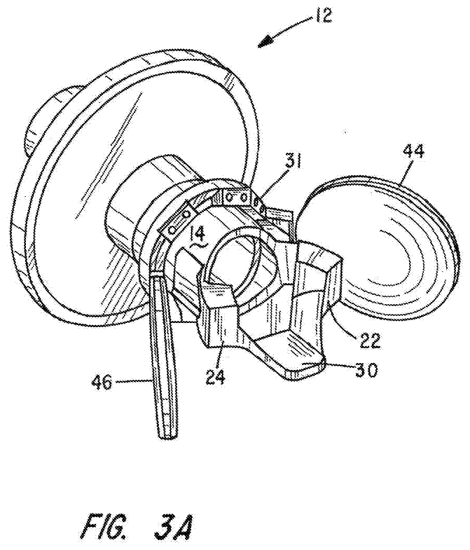

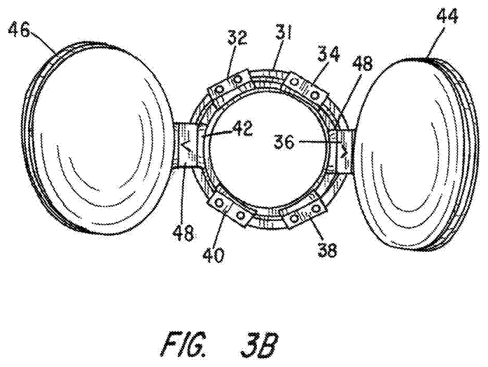

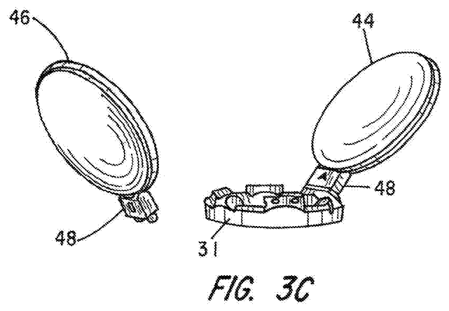

Turning next to FIG. 3A-3C, there is disclosed thereon a cheek support to be used during FOT tests to reduce cheek vibrations that can adversely affect the measurement of airway impedance. The cheek support comprises a toroidal base member 31 having a plurality of pairs of diametrically opposed connector receptacles as at 32, 34, 36, 38, 40 and 42. As will be explained, these receptacles permit cheek-engaging members 44, 46 to be positioned with differing spacing therebetween. To achieve this result, the connector receptacles include a pair of bores formed inwardly in a front surface 48 of the toroidal member 31. The bores comprising the several connector receptacles are formed so as to have differing angles of inclination relative to the front surface of the base member 30. The cheek-engaging members 44 and 46 each have connector portions, as at 48, to allow the coupling of a cheek-engaging member to a selected pair of diagonally positioned connector receptacles. In one embodiment, connector portions of the cheek-engaging members include a pair of pins adapted for insertion in the selected diagonally located pair of connector receptacles. In this embodiment, the pin-receiving bore formed in the connector portions 48 of the cheek supports are disposed off-center, thereby affording a further variation in the spacing between the cheek-engaging members 44 and 46, depending upon how the connectors 48 are oriented with respect to the receptacles elected for use on the toroid 30.

Without limitation, the annular ring 31 and the cheek-engaging members 44 and 46 are preferably a rigid plastic, such as a polycarbonate. The polycarbonate may be sandwiched between layers of neoprene pads for affording greater comfort to the user.

In use, the ring 31 with its cheek-engaging members 44 and 46 is mounted on the mouthpiece assembly of FIG. 1 in the manner shown in the view of FIG. 3A. the proximal end of the assembly is inserted in the subject's mouth and gripped in between the teeth, as earlier described. The members 44 and 46 will be initially set to press against the subject's cheeks to stabilize them as pulsatile air pressure changes are superimposed on a normal respiratory airflow reaching the patient during the FOT test.

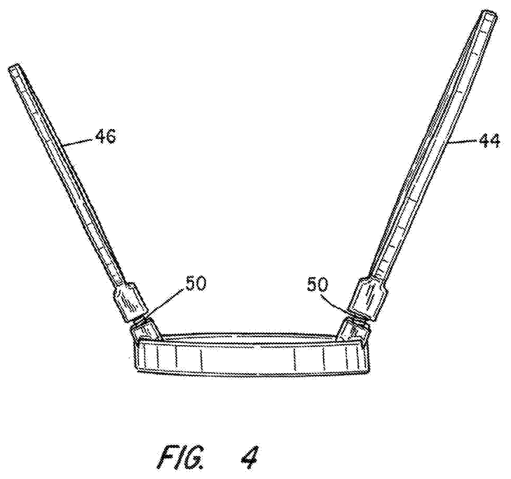

FIG. 4 shows an alternative embodiment of the cheek support member in which the pin arrangement illustrated in FIG. 3C are replaced by coil springs as at 50.

The mouthpiece of the present invention is adapted to be coupled to a FOT measuring instrument, such as a Resmon™ PRO Full device to measure the mechanical properties of a subject's lung using FOT. This device is available from MGC Diagnostics, Inc. of Vadnais Heights, Minn.

This invention has been described herein in considerable detail in order to comply with the patent statutes and to provide those skilled in the art with the information needed to apply the novel principles and to construct and use embodiments of the example as required. However, it is to be understood that the invention can be carried out by specifically different devices and that various modifications can be accomplished without departing from the scope of the invention itself.

What is claimed is:

1. A mouthpiece for delivering respiratory gas to a measuring instrument comprising:
a flexible, unitary, molded plastic member having a tubular portion of circular cross section with integrally molded wedge-shaped projections extending laterally outward from an outer wall of the tubular portion and circumferentially spaced from one another, the wedge-shaped projections leading to and integrally joined with a pair of curved block segments of predetermined heights and thickness dimensions that exceed a thickness of the tubular portion, and a generally rectangular, planar portion joined to the block segments by integrally formed, downwardly and inwardly sloping bridge segments.

2. The mouthpiece as in claim 2 wherein when an end portion of the mouthpiece is inserted into a subject mouth with the tubular portion of circular cross-section extending outward therefrom, and with the pair of curved blocks being clamped between the subject's upper and lower teeth, the generally rectangular, planar portion serves to depress the subject's tongue.

3. The mouthpiece of claim 1, when positioned as in claim 2, the wedge-shaped projections seal against the subject's lips at the corners of the subject's mouth.

4. The mouthpiece as in claim 1 wherein the plastic is silicone rubber.

5. The mouthpiece as in claim 1 and further including an anti-bacterial filter device connectable to the tubular portion of the mouthpiece.

6. The mouthpiece as in claim 1 and further including a cheek suppressor connectable to the tubular portion of the mouthpiece.

7. The mouthpiece as in claim 6 wherein the cheek suppressor comprises a toroidal mounting member having a plurality of pairs of diagonally located connector receptacles formed inwardly in a front surface of the toroidal member, said plurality of pairs of connector receptacles each having differing angles of inclination relative to the front surface of the toroidal mounting member, and a pair of cheek-engaging members each having a connector portion for coupling the cheek-engaging members to a selected pair of said connector receptacles.

8. The mouthpiece as in claim 7 wherein the connector portion of the cheek engaging members includes a pair of pins adapted for insertion in a selected pair of said connector receptacles.

9. The mouthpiece as in claim 7 wherein the connector portion of the cheek-engaging members includes at least one coiled spring adapted for insertion in a selected pair of said connector receptacles.

10. The mouthpiece as in claim 7 wherein the cheek engaging members comprise rigid plates with the connector portion extending out from a peripheral edge of the plates.

11. The mouthpiece as in claim 10 wherein the rigid plates are generally circular and comprise a polycarbonate plastic.

12. The mouthpiece as in claim 7 wherein the internal diameter of the toroidal member is sized to fit snugly about said tubular portion of the mouthpiece.

13. The mouthpiece as in claim 10 and further including neoprene pads adhered to the major surfaces of the rigid plates.
